(19) United States
(12) Patent Application Publication
(10) Pub. No.: US 2018/0214629 A1
(43) Pub. Date: Aug. 2, 2018

(54) Liquid Dispensing Port and Method and System Related Thereto

(71) Applicant: Poly Medicure Limited, Faridabad (IN)

(72) Inventors: Rishi Baid, New Delhi (IN); Amit Dogra, Faridabad (IN)

(73) Assignee: Poly Medicure Limited, Faridabad (IN)

(21) Appl. No.: 15/887,623

(22) Filed: Feb. 2, 2018

(30) Foreign Application Priority Data
Feb. 2, 2017 (IN) 201711003942

(51) Int. Cl. A61M 5/14; A61M 5/165; A61M 5/168

(52) U.S. Cl. CPC A61M 5/1411; A61M 2005/1652; A61M 5/16822; A61M 5/165

(57) ABSTRACT

A liquid dispensing port (10) comprising: a distal end (12) and a proximal end (14), wherein the distal end (12) is received in a container/bottle (16), an infusion tube (18) received in the proximal end (14); a holding rim (20) provided in between the proximal (14) and distal (12) end; axially extending openings (22) and (24) formed in the liquid dispensing port (10); an air vent (26) extending axially parallel to the openings (22) and (24) is provided in the port (10) which facilitates the passage of liquid from the container (16) through the tube (18).

### CROSS REFERENCE TO RELATED APPLICATIONS

This application claims priority to and benefit of Indian Patent Application No. 201711003942 filed Feb. 2, 2017. The contents of the above-identified application are relied upon and incorporated herein in their entirety by reference.

### FIELD OF THE INVENTION

The present invention relates to liquid dispensers adapted to be fitted to a container holding liquid. More particularly, the present invention relates to a liquid dispensing port and liquid dispensing system, and methods and system comprising the liquid dispensing port for dispensing liquid whereby a desired quantity of the liquid can be dispensed at a controllable flow rate without interruption.

The liquid dispensing port and liquid dispensing system can be used for dispensing liquid (for example glucose solution, saline solution, medicine, blood, blood components, etc.) to be infused into a human or animal body i.e. a patient by means of an infusion system.

### BACKGROUND OF INVENTION

Intravenous infusion system with a flow regulating and stopping liquid dispensers are already known and used to administer controlled quantities of intravenous fluid (for example glucose solution, saline solution, medicine, blood, blood components, etc.) into a patient's body seeking treatment. Such infusion systems are either pump driven or gravity driven infusion devices.

Gravity driven infusion devices typically include a drip chamber with or without a flow regulating and stopping member, a length of flexible infusion line to connect the lower end of the drip chamber to a standard connector. The connector is then connected via conventional access means such as a needle, a catheter or the like to a patient. The upper end of the drip chamber is provided with a spike which is used to pierce a fluid source such as a bag or bottle containing a specified infusion fluid.

However, various problems and disadvantages are associated with such infusion devices. One of the major problems commonly faced is that certain amount of air in the form of bubbles may get into the blood circulation of the patient accidently during the infusion procedure which may lead to air embolism. The air embolism in a patient may cause breathlessness, chest pain, stroke, wheezing, fast heartbeat, loss of consciousness or the like and may lead to death.

Some systems rely on a dip tube to draw the liquid out of containers of liquids which always leads to a small percentage of the liquid being wasted. Moreover, current systems relying on a dip tube to draw the liquid out of the container have problems with the exchange of the container: air bubbles can be trapped at the bottom of the tube which if not purged out, will cause process defects leading to losses as well as increased production costs.

It is desired to provide an improved liquid dispensing port adapted to be fitted to a container holding liquid, whereby a desired quantity of the liquid can be dispensed from the container at a controllable flow rate without interruption. It is also desired to provide an improved liquid dispenser port which is inexpensive to manufacture, efficient, effective and simple in its construction and use.

### SUMMARY OF INVENTION

A primary object and advantage of the present invention is to provide an improved liquid dispensing port which is easy to install and use.

Another object and advantage of the present invention is to provide an improved liquid dispensing port which has an ability to be used in many different types of containers with many different types of liquids and be easily cleaned.

Another object and advantage of the present invention is to provide an improved liquid dispensing port which is inexpensive to manufacture, efficient, effective and simple in its construction and use.

Another object and advantage of the present invention is to provide an improved liquid dispensing port which act as adapters that can be pressed and/or fitted into the open end of a container/bottle neck and/or opening to allow the use of dispenser which restricts leakage from the bottle and resists removal from the bottle.

Another object and advantage of the present invention is to provide a liquid dispensing port and liquid dispensing system, and methods and system comprising the liquid dispensing port which prevents the instances of air embolism.

Another object and advantage of the present invention is to provide a liquid dispensing port of unique design.

Another object and advantage of the present invention is to provide a liquid dispensing port and liquid dispensing system, and methods and system comprising the liquid dispensing port which helps reducing work load with respect to its installation and use.

Another object and advantage of the present invention is to provide a liquid dispensing port and liquid dispensing system, and methods and system comprising the liquid dispensing port which can help in preparing a series of infusions at once which is quicker and more hygienic than ever before.

Yet another object and advantage of the present invention is to provide a liquid dispensing port having means to communicate ambient air pressure to the liquid level surface in the liquid container. One of the means used is a filter which is permeable to air and impermeable to liquid being fitted in a channel provided in the liquid dispensing port. The filter is provided within the channel, preferably, at the distal end thereof. However, the filter can be arranged anywhere between the proximal and distal ends of the channel. The filter is designed such that it is permeable for air to pour out of the container into the atmosphere but prevents liquid contained in the container from pouring out through an opening into the atmosphere.

According to the invention, it is possible that the filter is formed by a hydrophobic or hydrophilic material. The filter is formed from hydrophobic material, i.e. material repelling water and other liquids. However, the filter material allows the passage of air.

Another object and advantage of the present invention is to provide a liquid dispensing port which is transferable from one container to another.

Yet another object and advantage of the present invention to provide a liquid dispensing port that may be easily assembled and disassembled without the use of tools.

One aspect of the present invention relates to an improved liquid dispensing port adapted to be fitted to a container holding liquid for example a bottle, whereby a desired quantity of the liquid can be dispensed from the container at a controllable flow rate without interruption. The liquid dispensing port comprising: a distal end and a proximal end, wherein the distal end is received in a container/bottle, an infusion tube is received in the proximal end; a holding rim is provided in between the proximal and distal end; axially extending openings formed in the liquid dispensing port; an air vent extending axially parallel to the openings is provided in the port which facilitate the passage of liquid from the container through the tube.

The air vent is provided with another axially extending openings to facilitate equalizing the pressure on the liquid level with ambient air pressure thereby allowing the liquid to freely flow.

Another aspect of the present invention relates to liquid dispensing system, and methods and system comprising the liquid dispensing port for dispensing liquid whereby a desired quantity of the liquid can be dispensed at a controllable flow rate without interruption.

In one embodiment, the liquid dispensing port is provided with a filter which is permeable to air and impermeable to liquid. Preferably the material of filter may be flexible and may be sheet or film, tube or fiber, or plug form or a combination thereof. The material may be a fabric, as a nonwoven, woven, or knit fabric, or a scrim. The filter is fitted in a channel provided in the liquid dispensing port.

In an embodiment, the liquid dispensing port can be constructed as a unitary dispensing port being a one-piece design. In an alternative embodiment, the liquid dispensing port can be molded with more than one piece.

In an embodiment, the liquid dispensing port which act as adapters that can be pressed and/or fitted into the open end of a bottle neck to allow the use of dispenser which restricts leakage from the bottle and resists removal from the bottle.

In an alternative embodiment, the liquid dispensing port can be fitted to a liquid container of the type having a cap threadably engaging external or male threads wherein the liquid dispensing port has internal or female threads such that the port can be threadably connected to the liquid container such as a one-gallon bottle.

### BRIEF DESCRIPTION OF FIGURES

The foregoing and other objects, features, and advantages of the invention will be apparent from the following detailed description taken in conjunction with the accompanying drawings, wherein:

FIG. 1 is a cross-sectional view of an exemplary embodiment of a liquid dispensing port of the present invention in its operative position inter-fitted into the top of a bottle.

FIG. 2 is a cross-sectional front view of a liquid dispensing port of the present invention.

FIG. 3 is a cross-sectional side view of liquid dispensing port of the present invention.

FIG. 4 is a perspective view of the liquid dispensing port of the present invention.

FIG. 5 is another perspective view of the liquid dispensing port of the present invention.

FIG. 6 is a front view of the liquid dispensing port of the present invention.

FIG. 7 is a side view of the liquid dispensing port of the present invention.

FIG. 8 is a top view of the liquid dispensing port of the present invention.

FIG. 9 is a bottom view of the liquid dispensing port of the present invention.

### DETAILED DESCRIPTION OF DRAWINGS

Generally speaking, the term “proximal” refers to a region of the device or parts thereof or a location on the device which is closest to, for example, a user using the device. In contrast to this, the term “distal” refers to a region of the device which is farthest from the user, for example, the distal region of a needle will be the region of a needle containing the needle tip which is to be inserted e.g. into a patients vein.

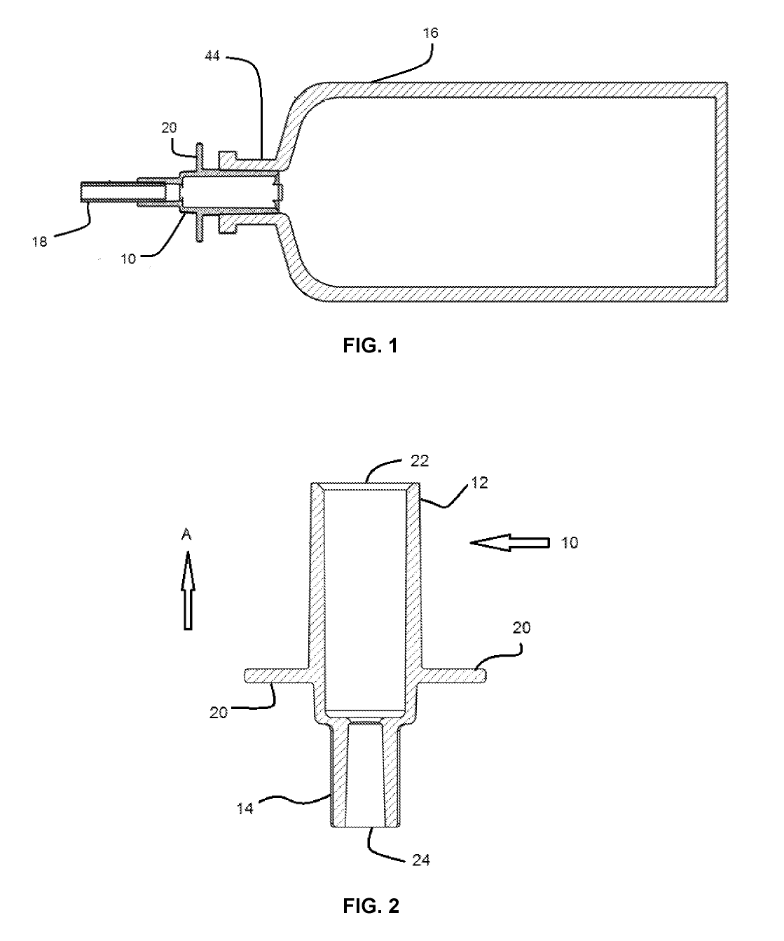

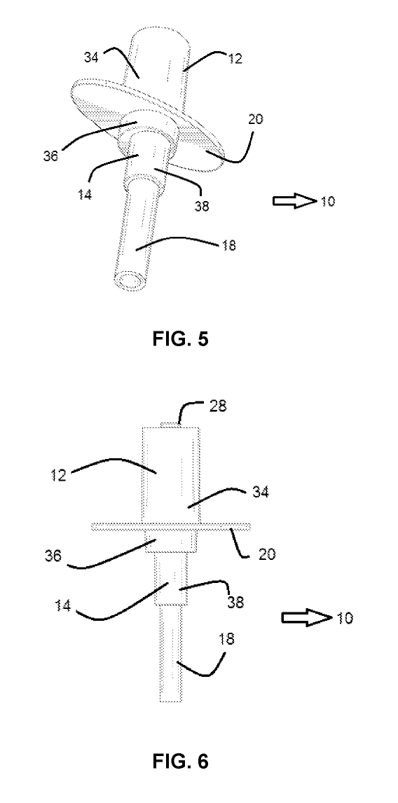

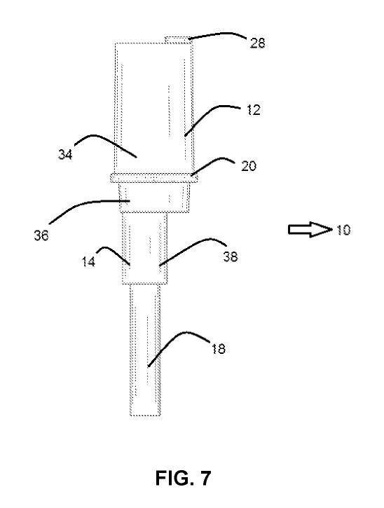

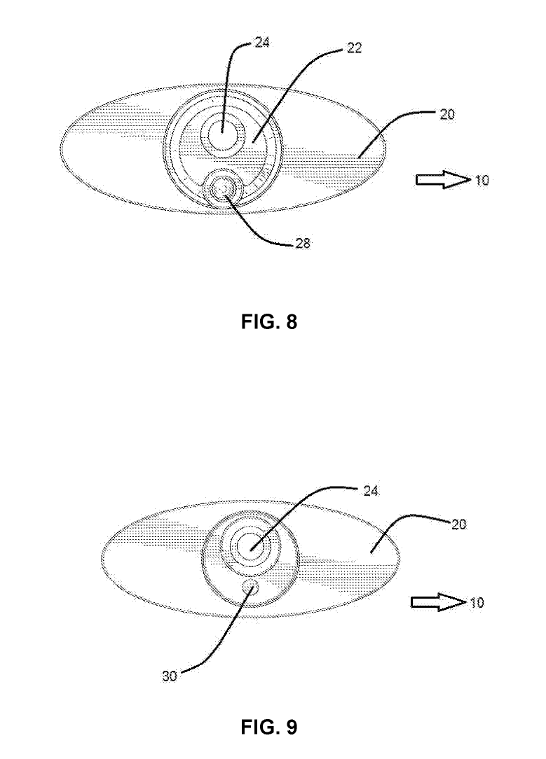

According to a first aspect of the present invention and referring to FIGS. 1 to 9, an exemplary embodiment of a liquid dispensing port of the present invention in its operative position inter-fitted into the top of a bottle 16. As illustrated in FIG. 1, the liquid dispensing port 10 comprises a distal end 12 and a proximal end 14. The distal end 12 is received in the mouth of the container/bottle 16. An infusion tube 18 of sufficient length is received in the proximal end 14 of the port 10 which connects to a standard connector (not shown) at the patient end so that a needle or a catheter could be connected to the patient in order to administer the liquid from the container 16. A holding rim 20 is provided in between the proximal 14 and distal 12 end of the liquid dispensing port 10 which facilitates the connection of the port 10 with the container 16. The holding rim 20 is received on the container 16, in particularly on the container's neck 44 such that it seats thereon and seals the container 16 and does not allow any spillage of liquid out of container 16. The container's neck includes an opening which allows the flow of liquid therefrom. Axially extending openings 22 and 24 are formed in the liquid dispensing port 10 for the passage of liquid from the container 16. Opening 22 communicates with the liquid in container 16 when the port is connected with the container 16 and opening 24 communicates through tube 18. The openings 22, 24 allow the liquid to pass through the tube 18 when the port 10 is connected to the container 16.

Referring now to FIGS. 4 to 7, the diameter of a top end 34 of liquid dispensing port 10 adjoining the holding rim 20 extending in the side of distal end 12 is greater than the diameter of a bottom end 36 adjoining the holding rim 20 on the other side extending in the side of proximal end 14. The liquid dispensing port 10 also has a lower end 38 adjoining the bottom end 36. The diameter of lower end 38 is further reduced from the diameter of the bottom end 36. The difference in diameters of top 34, bottom 36 and lower 38 end ensures a smooth flow of liquid out of container 16 in use.

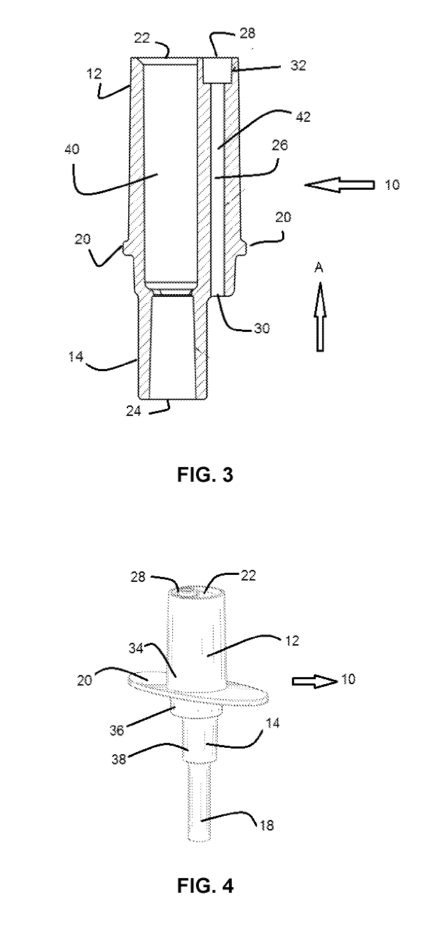

As illustrated in FIG. 3, an air vent 26 extending axially parallel to the openings 22 and 24 is provided in the port 10. The air vent 26 is also provided with another axially extending openings 28 and 30 which facilitate equalizing the pressure on the liquid level with ambient air pressure thereby allowing the liquid to freely flow. A filter 32 is arranged proximal to the opening 28 which communicates with the liquid in container 16 when the port 10 is connected with the container 16.

Thus, the port 10 comprises two flow paths (i) a first passage/channel 40 formed by axially extending openings 22 and 24 that allows liquid to pass out of the container 16; and (ii) a second passage/channel 42 formed by axially extending openings 28 and 30 to allow air to enter or pass-through the container 16.

The first passage/channel 40 facilitates only the passage of liquid from the container 16 and does not allow air flow due to the second passage/channel 42 which runs parallel to each other and do not share any connection. This independent parallel arrangement does not hinder the passage of free flow of liquid from the container 16 when in use. This parallel arrangement ensures that a person pouring liquid from container 16 do not face the problem of liquid flow erratically and the problem of “glugging” i.e. uneven flow of liquid caused by unbalanced pressures between the atmospheric air pressure outside the container 16 and the air pressure within the container 16. The port 10 not only minimizes or prevents the common glugging effect but it can allow liquid from a container 16 to be poured smoothly at any angle.

The filter 32 is preferably permeable to air and impermeable to liquid. In an alternative embodiment, the filter 32 can be arranged anywhere in between the openings 28 and 30. In one embodiment the filter 32 is cylindrical in shape with a larger diameter on its side falling in the container 16 than an inner diameter within the openings 28 and 30. Other shape of filter 32 such as oval, square, semi-circular, rectangular or the like can also be used.

Having a cylindrical filter 32 with a larger diameter on its side falling in the container 16 is advantageous, because a certain blockage of the filter 32, if occurs, is compensated by the diameter difference, such that a continuous flow of air through the filter 32 and to the atmosphere is still ensured.

A fluid flow adjustment device such as a roller clamp (not shown) can be arranged between the tube 18 and the standard connector being movable along the length of the flexible infusion tube 18. The roller clamp could be gradually adjusted so that the infusion tube could be squeezed proportionally to change the infusion rate and may also help in changing the fluid source i.e. bottle or container for multiple infusion procedures.

A clip (not shown) may also be arranged between the on the flexible tube 18 along its. The clip is configured to stop the flow of infusion fluid instantly by a clipping action.

The filter 32 can be sealed in the air vent by heat sealing, adhesive sealing, ultrasonic welding, heated die, radio frequency, mechanical seal, insert molding, laser welding, press/snap fit, annular ring with groove fitment etc. The filter 32 has a strong affinity for air while restricting liquid to pass through its surface. As a result, the filter 32 is permeable to air and impermeable to fluid/liquid which facilitate equalizing the pressure on the liquid level with ambient air pressure thereby allowing the liquid to freely flow.

Furthermore, because the same liquid dispensing port 10 can be used for multiple infusions, the material cost and clinical waste is greatly reduced and this is one of the advantages of the present invention.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. Thus, from the foregoing description, it will be apparent to one of ordinary skill in the art that many changes and modifications can be made thereto without departing from the spirit or scope of the invention as set forth in the claims.

Accordingly, it is not intended that the scope of the foregoing description be limited to the exact description set forth above, but rather that such description be construed as encompassing such features that reside in the present invention, including all the features and embodiments that would be treated as equivalents thereof by those skilled in the relevant art.

Thus, it is intended that the scope of the present invention herein disclosed should not be limited by the particular disclosed embodiments described above but should be determined only by a fair reading of the appended claims.

### LIST OF REFERENCE NUMERALS

10 liquid dispensing port
12 distal end
14 proximal end
16 container/bottle
18 infusion tube
20 holding rim
22, 24 openings
26 air vent
28, 30 openings
32 filter
34 top end
36 bottom end
38 lower end
40 first passage/channel
42 second passage/channel
44 neck

What is claimed is:

1. A liquid dispensing port comprising:
a distal end and a proximal end, wherein the distal end is received in a container;
an infusion tube received in the proximal end;
a holding rim provided in between the proximal end and the distal end;
first and second axially extending openings formed in the liquid dispensing port;
an axially extending air vent parallel to the first and second openings, which facilitates the passage of liquid from the container through the infusion tube.

2. The liquid dispensing port as claimed in claim 1, wherein first axially extending opening communicates with the liquid in the container when the liquid dispensing port is connected with the container and second axially extending opening communicates through the infusion tube.

3. The liquid dispensing port as claimed in claim 1, wherein the air vent is provided with third and fourth axially extending openings to facilitate equalizing the pressure on the liquid level with ambient air pressure thereby allowing the liquid to freely flow.

4. The liquid dispensing port as claimed in claim 3, wherein a filter is arranged proximal to the third axially extending opening which communicates with the liquid in container when the liquid dispensing port is connected with the container.

5. The liquid dispensing port as claimed in claim 4, wherein the filter is permeable to air and impermeable to liquid.

6. The liquid dispensing port as claimed in claim 1, wherein the diameter of a top end of the liquid dispensing port, which adjoins a distal side of the holding rim and extends in a direction of the distal end is greater than the diameter of a bottom end of the liquid dispensing port, which adjoins a proximal side of the holding rim and extends in a direction of the proximal end.

7. The liquid dispensing port as claimed in claim 1, further comprising a lower end of the liquid dispensing port, which adjoins the bottom end, and wherein the diameter of lower end is further reduced from the diameter of the bottom end.

8. The liquid dispensing port as claimed in claim 1, further comprising: a first flow path comprising a first channel that allows liquid to pass out of the container and is formed by the first and second axially extending openings; and a second flow path comprising a second channel to allow air to enter the container and is formed by third and fourth axially extending openings.

9. The liquid dispensing port as claimed in claim 4, wherein the filter is cylindrical in shape with a larger diameter on a side falling in the container than an inner diameter on a side within the third and fourth openings.

10. The liquid dispensing port as claimed in claim 4, wherein a shape of the filter may be oval, square, semi-circular, or rectangular.

11. The liquid dispensing port as claimed in claim 1, wherein the holding rim is received on the container, in particularly on a neck of the container.

12. An intravenous infusion system comprising:
a container holding a liquid;
a liquid dispensing port; and
an infusion tube;
wherein the liquid dispensing port communicates the liquid between the container and the infusion tube; and
wherein the liquid dispensing port comprises a first channel for communicating the liquid and a second channel for equalizing pressure in the container with an ambient pressure.

13. The intravenous infusion system of claim 12, wherein the first channel and the second channel are generally parallel; and wherein the first channel and the second channel have no direct connection therebetween within the liquid dispensing port.

14. The intravenous infusion system of claim 13, wherein the second channel has a smaller volume than a volume of the first channel; and wherein the second channel has a filter.

15. The intravenous infusion system of claim 14, wherein the filter in the second channel is hydrophobic.

16. The intravenous infusion system of claim 15, wherein the filter is secured in the second channel by heat sealing, adhesive sealing, ultrasonic welding, heated die, radio frequency, mechanical seal, insert molding, laser welding, interference fit, or annular ring with groove fitment.

17. The intravenous infusion system of claim 15, wherein a material of the filter is flexible and is provided in a sheet, film, tube, fiber, or plug form, or a combination thereof.

18. The intravenous infusion system of claim 15, wherein a material of the filter is nonwoven fabric, woven fabric, knit fabric, scrim, or a combination thereof.

19. The intravenous infusion system of claim 15, wherein the first channel and the second channel are perpendicular to the ground such that the container is in a position above the liquid dispensing port and the infusion tube is in a position below the liquid dispensing port.

20. The intravenous infusion system of claim 19, wherein the liquid dispensing port has a larger diameter on an end closest to the container and a smaller diameter on an end closest to the infusion tube.
